(19) United States
(12) Patent Application Publication
(10) Pub. No.: US 2010/0196286 A1
(43) Pub. Date: Aug. 5, 2010

(54) INHALATION DELIVERY METHODS AND DEVICES

(76) Inventors: Thomas A. ARMER, Cupertino, CA (US); Robert O. Cook, Hillsborough, NJ (US); Terry Alfredson, San Mateo, CA (US); Anthony Sun, New York, NY (US)

Correspondence Address: MORRISON & FOERSTER LLP, 755 PAGE MILL RD, PALO ALTO, CA, 94304-1018, US

(21) Appl. No.: 12/628,951

(22) Filed: Dec. 1, 2009

Related U.S. Application Data
(60) Provisional application No. 61/119,015, filed on Dec. 1, 2008.

(51) Int. Cl. A61K 31/395; A61K 31/4164; A61K 31/4168; A61K 31/165; A61K 31/155; A61K 31/54; A61K 31/433; A61K 9/12; A61K 31/5517; A61K 31/5513; A61K 31/519; A61K 31/4995; A61K 31/343; A61K 31/404; A61K 31/472; A61K 31/553; A61K 31/506; A61P 25/22; A61P 25/24; A61P 25/28

(52) U.S. Cl. 424/45; 514/396; 514/401; 514/617; 514/632; 514/183; 514/227.2; 514/362; 514/220; 514/221; 514/259.31; 514/249; 514/468; 514/419; 514/307; 514/211.13; 514/252.15

(57) ABSTRACT

Methods, devices and kits for treating sleep disorders, anxiety disorders, and developmental disorders, and/or for inducing an arousable state of sedation in a subject, are described. For example, inhalation methods, devices and kits for treating insomnia, anxiety and/or ADHD using one or more α2-adrenergic agonists such as dexmedetomidine or clonidine are described.

### CROSS-REFERENCE TO RELATED APPLICATIONS

This application claims the benefit of U.S. Provisional Application No. 61/119,015, filed Dec. 1, 2008, the disclosure of which is herein incorporated by reference in its entirety.

### FIELD

Methods and devices for treating sleep disorders, anxiety disorders, and/or developmental disorders, and/or for inducing an arousable state of sedation in a subject (e.g., for a perioperative, diagnostic, or non-surgical procedure), are described herein. More specifically, inhalation methods and devices for inducing an arousable state of sedation in a subject, and/or for treating insomnia (e.g., MOTN insomnia), anxiety, and/or attention deficit disorders (e.g., Attention-Deficit Hyperactivity Disorder, or ADHD) using one or more α2-adrenergic agonists (e.g., dexmedetomidine, medetomidine, clonidine, guanfacine, etc.) are described herein.

### BACKGROUND

Sleep disorders (e.g., insomnia), anxiety disorders (e.g., panic disorder), and developmental disorders (e.g., Attention-Deficit Hyperactivity Disorder, or ADHD) affect millions of people. For example, it is estimated that insomnia affects about 60-70 million Americans. Sleep, anxiety, and developmental disorders can have a significant negative impact on quality of life, compromising the health, general well-being, and/or safety of the person suffering from the disorder. Attention deficit disorders, e.g., ADHD, affect both adults and children, lowering their quality of life and productivity. In some instances, a person may suffer from more than one of these disorders. For example, a person may suffer from both a sleep disorder and an anxiety disorder (e.g., with one disorder causing the other).

Insomnia involves a persistent inability to fall asleep or persistent difficulty in falling asleep and/or remaining asleep during normal sleep times. A persistent inability to fall asleep is more specifically referred to as sleep onset insomnia, while the inability to remain asleep is more specifically referred to as sleep maintenance insomnia. Insomnia may be transient (e.g., lasting for days to weeks), acute (e.g., lasting for several weeks to several months), or chronic (e.g., lasting for years). Insomnia may be caused by, for example, certain drugs and/or stimulants (e.g., caffeine), hormonal fluctuations, stress, anxiety, depression, and/or neurological disorders, among other factors.

Insomnia may be treated using drugs in the form of pills, capsules, fast-melt tablets or injections. For example, sedative hypnotic drugs such as benzodiazepines have been used to treat insomnia for many years. Examples of benzodiazepines include temazepam (e.g., Restoril®), flunitrazepam (e.g., Rohypnol®), triazolam (e.g., Halcion®), flurazepam (e.g., Dalmane®), nitrazepam (e.g., Mogadon®), and midazolam (e.g., Versed®). Non-benzodiazepine agents also have been used to treat insomnia and include, for example, zolpidem (e.g., Ambien® and Ambien CR®), zaleplon (e.g., Sonata®), and eszopiclone (e.g., Lunesta®). In some cases, the antihistamine diphenhydramine (e.g., Benadryl®) has been used as a sleep aid. Diphenhydramine is available over the counter and does not seem to induce dependence, but its effectiveness may decrease over time. Additionally, it may result in next-day sedation.

One specific form of insomnia is middle-of-the-night insomnia (or “MOTN insomnia”). People suffering from MOTN insomnia experience difficulty returning to sleep after awakening in the middle of their normal sleep period (which, it should be noted, may not necessarily be at night (e.g., as in the case of a shift worker)). While they may not have problems initially falling asleep, they wake up prior to their intended wake time. Because it disrupts normal sleep patterns, MOTN insomnia can result in fatigue following the normal sleep period. One or more of the above treatments may be unsuitable for treating MOTN insomnia. For example, the treatment or treatments may be slow to induce sleep and/or may require administration prior to about seven to nine hours in bed to avoid residual sleepiness after the normal wake-up time. Also, some of the above-described hypnotics may be administered prophylactically, which may result in unnecessary medication and/or overmedication. Moreover, it may be undesirable to have to self-administer a pill or injection in the middle of the night to treat insomnia.

Another common form of insomnia is sleep onset insomnia, which is estimated to afflict approximately 10% of the population in the United States. A person suffering from sleep onset insomnia is not able to fall asleep upon retiring. The sleepless person may be restless (often for hours) and/or anxious, and may experience mental processing of daily activities which exacerbates the insomnia. Sleep onset may be induced by taking one or more medications in advance of retiring (e.g., one hour prior to retiring). However, this may interfere with the person's evening schedule or routine. Moreover, taking such medications in advance of retiring has led to morbidity, particularly in the form of hip fractures from drowsiness-induced falls.

Sleep maintenance insomnia is another form of sleeplessness. In the case of sleep maintenance insomnia, the subject has difficulty falling asleep and remaining asleep for a prolonged period of normal sleep cycles. This type of insomnia leaves the subject chronically fatigued and unable to restore normal sleep patterns. Many of the therapeutic agents used to treat sleep maintenance insomnia have long half-lives, and therefore have long duration of effect (e.g., usually 5-6 or more hours of non-arousable sedation). Such long duration of effect can result in a morning “hangover”, with associated reduction in cognitive ability during waking hours. Moreover, the use of long-acting agents may result in disrupted sleep quality (sleep cycle disruption), and may also cause sleep-walking, sleep-eating and/or sleep-driving. While some benzodiazepine agents with short half-lives may be used, they can precipitate withdrawal symptoms, in addition to causing amnesia and/or habitualization. In view of the above, it would be desirable to provide a rapidly-acting agent that does not have a prolonged duration of effect, and that has minimal adverse side effects. It would be particularly desirable to provide a rapidly-acting sleep maintenance agent that has minimal or no adverse effect on sleep cycle, and that does not provide a waking hour sedative hangover.

Anxiety disorders are psychological and/or physiological disorders that may result in a person experiencing anger, fear, apprehension, and/or worry. Anxiety may result in physical effects, such as heart palpitations, nausea, chest pain, shortness of breath, stomachaches, headaches, sweating, trembling, diarrhea, chills, pupillary dilation, hot flashes, sudden tiredness, hypertension, and/or digestion problems. There are many different types of anxiety disorders, including generalized anxiety disorder, panic disorder, phobias such as agoraphobia, social anxiety disorder, obsessive-compulsive disorder, post-traumatic stress disorder, and separation anxiety disorder. An additional form of anxiety is hospital procedural anxiety, such as that experienced by a patient prior to a procedure (e.g., needle use, magnetic resonance imaging (MRI) scanning, etc.). Some anxiety sufferers experience panic attacks, which usually come with little or no warning. A person experiencing a panic attack may suffer from headaches, heart palpitations, dizziness, insomnia and/or chest pain, and may feel as if he or she is about to faint or even die. Panic disorder may make it very difficult or even impossible to cope with normal daily activities.

A number of drugs, also known as anxiolytics, may be prescribed to treat anxiety disorders. Anxiolytics are generally divided into two groups of medication: benzodiazepines and non-benzodiazepines. Examples of benzodiazepines that can function as anxiolytics include lorazepam (e.g., Ativan®), clonazepam (e.g., Klonopin®), alprazolam (e.g., Xanax®), and diazepam (e.g., Valium®). Though not anxiolytics, beta-receptor blockers (e.g., propranolol, oxprenolol) may also be used to treat anxiety. Additionally, atypical antipsychotics such as loxapine, doxepin, and serotonin agonists such 5HT-2a antagonists have recently been applied for treatment. Other treatments of anxiety disorders may, involve herbs, psychotherapy, and/or lifestyle changes.

Developmental disorders include, for example, learning disabilities and neurodevelopmental disorders such as ADHD. ADHD is a poorly defined behavioral syndrome that is characterized by short attention span, hyperkinetic physical behavior and learning problems. It is believed that ADHD may be hereditary in some cases, but that it may also sometimes be caused by other factors (e.g., trauma). A person with ADHD may have trouble controlling his or her impulses, and may have difficulty with concentration, memory, and/or organization. In certain instances, a person suffering from ADHD may also suffer from an anxiety disorder.

ADHD may be treated, for example, with one or more medications, by implementing lifestyle changes, and/or by receiving counseling. Examples of medications that may be used to treat ADHD include stimulant medications, such as methylphenidate (e.g., Ritalin®), dextroamphetamine (e.g., Dexedrine®), a mixture of amphetamines (e.g., Adderall®), and guanfacine (e.g., Tenex®). Generally, treatment of ADHD is characterized by the prophylactic administration of long-acting medication to provide continuous therapy throughout the day. This may result in numerous adverse effects, such as palpitations, feeling faint, significant blood pressure effects, aggression, restlessness, hallucinations and/or unusual behavior. Accordingly, it would be advantageous to provide a rapid-acting and safe medication which can be simply administered to or by a subject at the onset of symptoms.

In view of the prevalence of sleep, anxiety, and developmental disorders in the world population, it would be desirable to provide additional compositions and methods that may be used to treat these disorders. It would be especially desirable to provide methods and compositions for treating sleep onset and MOTN insomnia that initiate sleep quickly, enable arousability, minimize the side effects associated with long duration sedatives, and/or do not involve administration by injection or pills. Additionally, a rapid-acting medication that could induce sleep or sedation within about 30 minutes or less of administration may avoid the schedule limitations of current remedies, while minimizing the potential for drowsiness-induced injury. Such medication could be taken immediately before or after the person has retired for the night, thus assuring that the person is already in bed when the sedation takes effect.

It would also be desirable to be able to induce an arousable state of sedation in a subject (e.g., for a perioperative, diagnostic and/or non-surgical procedure). A method of inhaled sedation can be especially useful prior to surgical procedures for pediatric patients. Initial levels of sedation can be achieved quickly without the frightening and painful steps of starting an IV or placing a mask on the face prior to intubation and should allow the child to be moved to the procedure suite or away from their parents with less anxiety. Once the pediatric patient is sedated methods to effectuate deeper levels of sedation can be implemented more quickly, safely and with less trauma for the patient. Inhaled sedation can also be helpful in achieving fast onset sedation for diagnostic and interventional procedures such as MRI, CT scans, wound debridement, abscess drainage, minor skin procedures, difficult vascular access or blood draws, laceration repairs, foreign body removal, endoscopy, colonoscopy, audiology ABR/BAER testing, intra ocular pressure testing, injections of the muscles, bursa, tendons or soft tissue, appliance removal, fracture reduction, ECHO testing, lumbar punctures and bone marrow aspiration procedures, or during nuclear medicine, fluoroscopy or interventional radiology procedures, difficult vascular access, EEG/EMG.SSEP procedures or dental surgery for children. Such a sedation method is also effective for adults and adolescents undergoing the aforementioned procedures when arousable, conscious sedation is required.

### SUMMARY

Methods, devices and kits for treating sleep disorders (e.g., insomnia), anxiety disorders (e.g., panic disorder), and developmental disorders (e.g., ADHD), and/or for inducing an arousable state of sedation in a subject, are described herein. Generally, the methods may comprise administering one or more α2-adrenergic agonists via inhalation. Specific examples of α2-adrenergic agonists include dexmedetomidine, medetomidine, detomidine, guanfacine and clonidine, although other suitable α2-adrenergic agonists may alternatively or additionally be used. In some variations, the α2-adrenergic agonist or agonists may be combined with one or more other therapeutic agents, such as a long-acting sedative-hypnotic. Methods described herein may comprise administering a therapeutically effective amount of an α2-adrenergic agonist by oral or nasal inhalation, such that there is rapid onset of action with minimal adverse side effects (e.g., undesirable central nervous system effects including diminished cognition and excessive, prolonged sedation, and sleep pattern disruption). In some variations, the α2-adrenergic agonists may be administered using inhalation devices that include an aerosol spray generating mechanism and an α2-adrenergic agonist composition. An inhalation device may be in the form of a pressurized metered dose inhaler (pMDI), a dry powder inhaler (DPI), or a nebulizer, for example. Additional devices that may be employed with one or more of the methods described herein include nasal or sublingual spray actuators. Devices used with the methods described here may be breath-actuated, and/or may be electronically, mechanically or pneumatically operated.

In addition to comprising one or more α2-adrenergic agonists, such as dexmedetomidine and/or clonidine, the compositions described here may include one or more excipients, such as propellants, carrier media, surfactants, stabilizers, flocculating agents, thickening agents, adhesive agents, absorption enhancers, solvents, dispersants, preservatives, antioxidants, buffering agents, and/or flavoring agents. Depending on the type of inhalation device employed, the α2-adrenergic agonist composition may be contained within a pressurized canister, blister, capsule, ampoule, spray dispenser, etc., or provided as a solid, which can be scraped, ground, crushed, pulverized, or the like, to form particles.

As described above, the inhalation devices may be used to treat various sleep, anxiety, and/or developmental disorders. For example, they may be used to treat insomnia, including acute insomnia, chronic insomnia, sleep onset insomnia, and sleep maintenance insomnia. In some variations, they may be used to treat MOTN insomnia. In certain variations, the inhalation devices may be used to treat panic disorder, and/or one or more other anxiety disorders, such as agoraphobia, social anxiety disorder, obsessive-compulsive disorder, post-traumatic stress disorder, and/or separation anxiety disorder. In some variations, the inhalation devices may be used to treat ADHD, and/or hyperactivity in both children and adults. In certain variations, they may be used to induce an arousable state of sedation within a subject.

In some variations, an α2-adrenergic agonist aerosol generated by the inhalation devices may be capable of rapidly initiating sleep, thus decreasing sleep latency. As used herein, the terms “rapid” or “rapidly” refer to the induction of sleep within about 30 minutes or less after administration of the α2-adrenergic agonist composition. In certain variations, the α2-adrenergic agonist aerosol may be capable of maintaining sleep for at least about two to three hours.

Initial levels of sedation may be achieved relatively quickly, without the frightening and painful steps of starting an IV or placing a mask on the face prior to intubation. In some variations, an arousable state of sedation may be induced that allows a child to be moved to the procedure suite or operating room away from their parents with less anxiety. Once the pediatric patient is sedated, methods to effectuate deeper levels of sedation may be implemented more quickly, safely and with less trauma for the patient. Inhaled sedation may also be helpful in achieving fast onset sedation for diagnostic and interventional procedures such as MRI, computed tomography (CT) scans, wound debridement, abscess drainage, minor skin procedures, difficult vascular access or blood draws, laceration repairs, foreign body removal, endoscopy, colonoscopy, audiology ABR/BAER testing, intraocular pressure testing, injections of the muscles, bursa, tendons or soft tissue, appliance removal, fracture reduction, echocardiography (ECHO) testing, lumbar punctures and bone marrow aspiration procedures, or during nuclear medicine, fluoroscopy or interventional radiology procedures, EEG/EMG.SSEP procedures or dental surgery for children. Such a sedation method may also be effective for adults and adolescents undergoing the aforementioned procedures when arousable, conscious sedation is required.

In further variations, a combination composition may be administered. The combination composition may comprise, for example, one or more α2-adrenergic agonists (e.g., dexmedetomidine, clonidine) and one or more other therapeutic agents. As an example, a composition may comprise one or more α2-adrenergic agonists to provide rapid induction of sleep, as well as one or more further therapeutic agents capable of providing long-acting sedation and/or enhanced sleep quality. The presence of an α2-adrenergic agonist in a combination composition used to treat insomnia may allow for a lower amount of other therapeutic agents to be included in the composition to provide sedation. For example, when eszopiclone is administered in combination with dexmedetomidine, the amount of eszopiclone required to provide a sedative effect may be lower than the amount of eszopiclone required when the eszopiclone is used by itself. Accordingly, the occurrence of undesirable side effects may be reduced, while also providing longer duration of sedation than with a non-combination composition, such as dexmedetomidine alone.

They may also be packaged as kits including an inhalation device and one or more α2-adrenergic agonist compositions. The inhalation devices may be disposable, single-use or multiple-use devices. Here the compositions may each provide the α2-adrenergic agonist in different doses. The kits may also be tailored to the type of sleep, anxiety or developmental disorder being treated.

Some variations of the methods and devices described here may provide for rapidly-acting sleep maintenance with minimal or no adverse effects on normal sleep cycle, and no sedative hangover upon waking. Because the compositions may take effect rapidly, they may be used just prior to retiring (for sleep onset) and again in the middle of the night (to return to sleep), without morning hangover effects. Such rapid action may not disrupt sleep patterns, (e.g., may take effect within about 30 minutes or less of retiring), and may have the benefit of increasing total sleep time and quality. It therefore may contribute towards sleep maintenance.

In one aspect, a method of treating a sleep disorder and/or inducing an arousable state of sedation in a subject comprises administering a therapeutically effective amount of an α2-adrenergic agonist composition to the subject using an inhalable or other non-injectable route of administration, to initiate an arousable state of sedation within the subject in about 30 minutes or less, where the α2-adrenergic agonist composition comprises an α2-adrenergic agonist or a pharmaceutically acceptable salt, hydrate, polymorph, prodrug, ion pair, or metabolite thereof.

In a further aspect, a method of treating insomnia in a subject comprises administering a therapeutically effective amount of a composition to the subject via an inhalable or other non-injectable route of administration to initiate an arousable state of sedation within the subject in about 30 minutes or less, where the composition comprises dexmedetomidine or clonidine.

In another aspect, a kit for use in treating a sleep disorder in a subject comprises at least one dose of an α2-adrenergic agonist composition comprising an α2-adrenergic agonist or a pharmaceutically acceptable salt, hydrate, polymorph, prodrug, ion pair, or metabolite thereof, where the at least one dose when administered to a subject using an inhaled or other non-injectable route of administration initiates an arousable state of sedation within the subject in about 30 minutes or less.

In a further aspect, a method of initiating an arousable state of sedation by comprises administering a therapeutically effective amount of a composition to the a subject via an inhalable or other non-injectable route of administration, where the method is part of an overall perioperative or non-surgical procedure, and the composition comprises dexmedetomidine, medetomidine, detomidine, guanfacine, or clonidine.

In an additional aspect, a method of treating an anxiety disorder in a subject comprises administering a therapeutically effective amount of an α2-adrenergic agonist composition to the subject using an inhalable or other non-injectable route of administration, to achieve therapeutic plasma levels in about 30 minutes or less, as indicated by a reduction in anxiety symptoms, where the α2-adrenergic agonist composition comprises an α2-adrenergic agonist or a pharmaceutically acceptable salt, hydrate, polymorph, prodrug, ion pair, or metabolite thereof.

In a further aspect, a kit for use in treating an anxiety disorder in a subject comprises at least one dose of an α2-adrenergic agonist composition comprising an α2-adrenergic agonist or a pharmaceutically acceptable salt, hydrate, polymorph, prodrug, ion pair, or metabolite thereof, where the at least one dose when administered to a subject using an inhalable or other non-injectable route of administration results in a reduction in anxiety levels in about 30 minutes or less.

In another aspect, a method of treating ADHD in a subject comprises administering a therapeutically effective amount of an α2-adrenergic agonist composition to the subject using a non-injectable route of administration, to achieve a reduction in hyperactivity or associated symptoms thereof in about 30 minutes or less, where the α2-adrenergic agonist composition comprises an α2-adrenergic agonist or a pharmaceutically acceptable salt, hydrate, polymorph, prodrug, ion pair, or metabolite thereof.

In an additional aspect, a kit for use in treating ADHD in a subject comprises at least one dose of an α2-adrenergic agonist composition comprising an α2-adrenergic agonist or a pharmaceutically acceptable salt, hydrate, polymorph, prodrug, ion pair, or metabolite thereof, where the at least one dose when administered to a subject using an inhalable or other non-injectable route of administration results in a reduction in hyperactivity or associated symptoms thereof in about 30 minutes or less.

The α2-adrenergic agonist composition may comprise an α2-adrenergic agonist selected from the group consisting of dexmedetomidine, derivatives of dexmedetomidine medetomidine, detomidine, clonidine, romifidine, guanfacine, guanabenz, guanoxabenz, guanethidine, xylazine, detomidine, medetomidine, tizanidine, other imidazole derivatives, and pharmaceutically acceptable salts, hydrates, polymorphs, prodrugs, ion pairs, or metabolites thereof.

The α2-adrenergic agonist composition may be administered to the subject via inhalation (e.g., via oral inhalation, via nasal inhalation) or via another non-injectable route of administration. The α2-adrenergic agonist composition may be administered to the subject as an aerosol composition. The α2-adrenergic agonist composition may be administered to the subject using at least one of a pressurized metered dose inhaler, a breath-activated metered dose inhaler, a dry powder inhaler, or a nebulizer. The subject may self-administer the α2-adrenergic agonist composition.

The Tₘₐₓ for the administration of the α2-adrenergic agonist may about 30 minutes or less (e.g., less than about 20 minutes). For treatment of sleep disorders, anxiety, and ADHD, the plasma concentration of the α2-adrenergic agonist composition in the subject at about 15 minutes or less after administration may be in the range of about 0.0015 ng/mL to about 600 ng/mL. The α2-adrenergic agonist composition may be administered to the subject at a dosage of from about 0.01 μg/kg to about 300 μg/kg. For perioperative and/or non-surgical procedures, the plasma concentration of the α2-adrenergic agonist composition in the subject at about 15 minutes or less after administration may be in the range of about 0.0015 ng/mL to about 600 ng/mL and the α2-adrenergic agonist composition may be administered to the subject at a dosage in the range of about 0.02 μg/kg to about 500 μg/kg. The α2-adrenergic agonist composition may be administered to the subject as two separate doses. The method may comprise administering a second therapeutic agent to the subject. The second therapeutic agent may be administered to the subject prior to administration of the α2-adrenergic agonist composition. The subject may be a human or an animal.

The kit may further comprise at least one pMDI, nasal spray or buccal spray canister containing the α2-adrenergic agonist composition. The at least one canister may have a total volume of less than about 10 mL (e.g., less than about 6 mL) and/or more than about 1 mL. The at least one canister may comprise a primeless valve. Primeless valves are disclosed, for example, in co-pending Provisional Patent Application Ser. No. 61/080,213 (filed Jul. 11, 2008), the disclosure of which is hereby incorporated by reference in its entirety. The kit may comprise at least one inhalation device (e.g., at least one pressurized metered dose inhaler). The kit may further comprise instructions. At least one dose of the α2-adrenergic agonist composition may be suitable to be administered to the subject at a dosage of from about 0.01 μg/kg to about 500 μg/kg (e.g., from about 0.05 μg/kg to about 10 μg/kg, from about 0.1 μg/kg to about 10 μg/kg, from about 0.1 μg/kg to about 5 μg/kg, from about 0.2 μg/kg to about 5 μg/kg, from about 0.2 μg/kg to about 4 μg/kg, from about 0.25 μg/kg to about 4 μg/kg, about 2 μg/kg). The kit may further comprise a second therapeutic agent, such as a sedative, a sedative-hypnotic, or an anxiolytic (e.g., buspirone, propranolol, alprazolam, or clonazepam). When used to treat insomnia, the kit may comprise a benzodiazepine selected from the group consisting of alprazolam, diazepam, temazepam, flunitrazepam, triazolam, flurazepam, nitrazepam, and midazolam. Alternatively or additionally, the kit may comprise a non-benzodiazepine selected from the group consisting of zolpidem, zaleplon, zopiclone, eszopiclone, ramelteon, melatonin, almorexant, and eplivanserin. When used to treat ADHD, the kit may comprise methylphenidate, dextroamphetamine/amphetamine, dextroamphetamine, atomoxetine, loxapine, doxepin, or a 5HT-2a antagonist.

The subject may have insomnia (e.g., sleep onset insomnia, middle-of-the-night insomnia), and the arousable state of sedation may be induced to treat the insomnia. An arousable state of sedation may be initiated within the subject in about 30 minutes or less (e.g., about 20 minutes or less, about 15 minutes or less, about 10 minutes or less, about 5 minutes or less). The second therapeutic agent may comprise a sedative or a sedative-hypnotic. The second therapeutic agent may comprise a benzodiazepine selected from the group consisting of alprazolam, diazepam, temazepam, flunitrazepam, triazolam, flurazepam, nitrazepam, and midazolam. The second therapeutic agent may comprise a non-benzodiazepine selected from the group consisting of zolpidem, zaleplon, zopiclone, eszopiclone, ramelteon, melatonin, almorexant, eplivanserin, loxapine, doxepin, and a 5HT-2a antagonist.

The anxiety disorder may comprise panic disorder. The anxiety disorder may comprise agoraphobia, social anxiety disorder, obsessive-compulsive disorder, post-traumatic stress disorder, pre-procedural anxiety, and/or separation anxiety disorder. Reduction in anxiety levels may be achieved within the subject in about 30 minutes or less (e.g., about 15 minutes or less, about 10 minutes or less, about 5 minutes or less). The second therapeutic agent may comprise an anxiolytic, such as buspirone, propranolol, alprazolam, clonazepam, loxapine, doxepin, or a 5HT-2a antagonist.

The arousable state of sedation may be induced for or as part of a perioperative procedure, a prediagnostic procedure, or a non-surgical procedure in a clinical setting. The arousable state of sedation may be induced as part of a medical procedure selected from the group consisting of MRI, CT scans, wound debridement, abscess drainage, skin procedures (e.g., minor skin procedures), vascular access or blood draws (e.g., difficult vascular access or blood draws), laceration repairs, foreign body removal, endoscopy, colonoscopy, audiology ABR/BAER testing, intraocular pressure testing, injections of the muscles, bursa, tendons or soft tissue, appliance removal, fracture reduction, echocardiography (ECHO) testing, lumbar punctures and bone marrow aspiration procedures, radiology procedures such as nuclear medicine, fluoroscopy, interventional procedures, EEG/EMG.SSEP, and dental surgery (e.g., for children).

In treating ADHD, a reduction in hyperactivity or associated symptoms thereof may be achieved within the subject in about 30 minutes or less (e.g., about 20 minutes or less, about 15 minutes or less, about 10 minutes or less, about 5 minutes or less). The second therapeutic agent may comprise methylphenidate, dextroamphetamine/amphetamine, dextroamphetamine, atomoxetine, loxapine, doxepin, or a 5HT-2a antagonist.

### DETAILED DESCRIPTION

Described here are methods of treating sleep, anxiety and developmental disorders, and/or inducing an arousable state of sedation in a subject, by administering α2-adrenergic agonists using nasal or oral inhalation or buccal spray devices. Examples of sleep disorders that may be treated include insomnia, such as acute insomnia, chronic insomnia, sleep onset insomnia, and sleep maintenance insomnia. In some variations, MOTN insomnia may be treated. Non-limiting examples of anxiety disorders that may be treated include agoraphobia, social anxiety disorder, obsessive-compulsive disorder, post-traumatic stress disorder, pre-procedural anxiety, and/or separation anxiety disorder. A non-limiting example of a developmental disorder that may be treated is ADHD. The inhalation devices may generally be configured to include an aerosol-generating mechanism and an α2-adrenergic agonist composition, such as a dexmedetomidine, medetomidine, or detomidine composition, or a guanfacine or clonidine composition. In some variations, a combination composition may be used. In certain variations, the devices may include a dose counter and/or lock-out mechanism.

The methods, devices, and/or kits described herein may provide for fast, efficient treatment of insomnia, anxiety, and/or developmental disorders. As an example, a person suffering from sleep onset insomnia may simply administer a dexmedetomidine, medetomidine or detomidine composition, or a guanfacine or clonidine composition, via inhalation of an aerosol spray just prior to, or upon retiring for, a normal sleep period. Such treatment is non-invasive and easy to self-administer (e.g., because it does not require an injection) and the therapeutic onset of action is rapid. If the person were to awaken in the middle of the normal sleep period, an additional dose could be readily administered (e.g., without the need to get out of bed, or to get a glass of water to take an oral dosage form). The rapid therapeutic effect and ease of administration would enable a fast return to sleep, without deleterious side effects such as over-sedation, sedation hangover, or sleep pattern disruption. Moreover, dexmedetomidine, medetomidine, detomidine, guanfacine, and clonidine, acting on non-GABA receptors, could provide for safe treatment of sleep, anxiety and/or developmental disorders, without inducing drug dependency, or drug tolerance.

### I. DEVICES

The inhalation devices or other non-injectable devices may be of various designs, so long as they are capable of generating an aerosol of an α2-adrenergic agonist. The devices generally include a housing having a proximal end and a body portion. A mouthpiece or nosepiece will typically be positioned at the proximal end. In one variation, the device may be a dry powder inhaler (DPI) with the composition adjusted to generate a significant portion of the delivered dose in the respirable range (drug particles less than approximately 5 microns median aerodynamic diameter (MMAD)). In another variation, the inhalation device may be a pressurized metered dose inhaler (pMDI) with the composition adjusted to generate a significant portion of the delivered dose in the respirable range (free drug or drug contained in propellant droplets having sizes less than approximately 5 microns median aerodynamic diameter (MMAD). In some variations the pMDI or DPI can be fitted with nosepiece adapters to administer the drug laden dry powder or propellant to the nasopharynx. In other variations the pMDI and or DPI can be conventionally fitted with a mouthpiece, but the compositions may be adjusted to generate a significant portion of the delivered dose in the nonrespirable range (free drug particles or drug contained in propellant droplets greater than approximately 10 microns median aerodynamic diameter (MMAD)) so that most of the drug is deposited in the oropharynx.

Dry Powder Inhalers

In a DPI, the dose to be administered is stored in the form of a non-pressurized dry powder and, on actuation of the inhaler, the particles of the powder are inhaled by the subject. Similar to pMDIs, a compressed gas may be used to dispense the powder. Alternatively, when the DPI is breath-actuated, the powder may be packaged in various forms, such as a loose powder, cake or pressed shape in a reservoir. Examples of these types of DPIs include the Turbohaler™ inhaler (Astrazeneca, Wilmington, Del.) and Clickhaler® inhaler (Innovata, Ruddington, Nottingham, UK). When a doctor blade or shutter slides across the powder, cake or shape, the powder is culled into a flowpath whereby the patient can inhale the powder in a single breath. Other powders are packaged as blisters, gelcaps, tabules, or other preformed vessels that may be pierced, crushed, or otherwise unsealed to release the powder into a flowpath for subsequent inhalation. Typical of these are the Diskus™ inhaler (Glaxo, Greenford, Middlesex, UK), EasyHaler® (Orion, Expoo, Fla.), and Novohaler™ inhalers. Still others release the powder into a chamber or capsule and use mechanical or electrical agitators to keep the drug suspended for a short period until the patient inhales. Examples of this are the Exubera® inhaler (Pfizer, New York, N.Y.), Qdose inhaler (Microdose, Monmouth Junction, N.J.), and Spiros® inhaler (Dura, San Diego, Calif.).

Pressurized Metered Dose Inhalers (pMDIs)

pMDIs generally have two components: a canister in which the drug particles are stored under pressure in a suspension or solution form, and a receptacle used to hold and actuate the canister. The canister may contain multiple doses of the composition, although it is possible to have single dose canisters as well. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the drug particles are atomized, forming an aerosol. pMDIs generally use propellants to pressurize the contents of the canister and to propel the drug particles out of the receptacle outlet. In pMDIs, the composition is provided in liquid form, and resides within the canister along with the propellant. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluoroalkane (HFA) propellants.

In some instances, a manual discharge of aerosolized drug must be coordinated with inhalation, so that the drug particles are entrained within the inspiratory air flow and conveyed to the lungs. In other instances, a breath-actuated trigger, such as that included in the Tempo® inhaler (MAP Pharmaceuticals, Mountain View, Calif.) may be employed that simultaneously discharges a dose of drug upon sensing inhalation, in other words, the device automatically discharges the drug aerosol when the user begins to inhale.

Nebulizers

Nebulizers are liquid aerosol generators that convert bulk liquids, usually aqueous-based compositions, into mists or clouds of small droplets, having diameters less than 5 microns mass median aerodynamic diameter (MMAD), which can be inhaled into the lower respiratory tract. This process is called atomization. The bulk liquid contains particles of the therapeutic agent(s) or a solution of the therapeutic agent(s), and any necessary excipients. The droplets carry the therapeutic agent(s) into the nose, upper airways or deep lungs when the aerosol cloud is inhaled.

Pneumatic (jet) nebulizers use a pressurized gas supply as a driving force for liquid atomization. Compressed gas is delivered through a nozzle or jet to create a low pressure field which entrains a surrounding bulk liquid and shears it into a thin film or filaments. The film or filaments are unstable and break up into small droplets which are carried by the compressed gas flow into the inspiratory breath. Baffles inserted into the droplet plume screen out the larger droplets and return them to the bulk liquid reservoir. Examples include the PARI LC® Plus®, or Sprint® nebulizers, the Devilbiss PulmoAide® nebulizer, and the Boehringer Ingelheim Respimat® inhaler.

Electromechanical nebulizers use electrically generated mechanical force to atomize liquids. The electromechanical driving force is applied by vibrating the bulk liquid at ultrasonic frequencies, or by forcing the bulk liquid through small holes in a thin film. The forces generate thin liquid films or filament streams which break up into small droplets to form a slow moving aerosol stream which can be entrained in an inspiratory flow.

One form of electromechanical nebulizers is ultrasonic nebulizers, in which the bulk liquid is coupled to a vibrator oscillating at frequencies in the ultrasonic range. The coupling is achieved by placing the liquid in direct contact with the vibrator such as a plate or ring in a holding cup, or by placing large droplets on a solid vibrating projector (a horn). The vibrations generate circular standing films which break up into droplets at their edges to atomize the liquid. Examples include the DuroMist® nebulizer, Drive Medical's Beetle Neb® nebulizer, Octive Tech's Densylogic® nebulizer, and the John Bunn Nano-Sonic® nebulizer.

Another form of an electromechanical nebulizer is a mesh nebulizer, in which the bulk liquid is driven through a mesh or membrane with small holes ranging from 2 to 8 microns in diameter, to generate thin filaments which immediately break up into small droplets. In certain designs, the liquid is forced through the mesh by applying pressure with a solenoid piston driver (AERx®), or by sandwiching the liquid between a piezoelectrically vibrated plate and the mesh, which results in a oscillatory pumping action (EFlow®, AerovectRx, TouchSpray™). In a second type the mesh vibrates back and forth through a standing column of the liquid to pump it through the holes (AeroNeb®). Examples include the AeroNeb Go®, Pro®; PARI EFlow®; Omron 22UE®; and Aradigm AERx®.

Nasal/Oral Spray Pumps

Spray pumps consist of a chamber that holds a suspension or solution of the therapeutic agent(s) and appropriate excipients, and a pump. The pump draws a measured aliquot of the fluid up a dip tube, and then expels it through a nozzle to generate droplets generally greater than 10 microns MMAD. The nozzle is placed into the mouth or nostril and actuated to deliver a dose of the therapeutic agent to the mouth or nose. Excipients can include preservatives, absorption enhancers, flavoring, thickening and adhesive agents.

II. COMPOUNDS

α2-Adrenergic Agonists

As described above, the compounds used in the composition described herein include α2-adrenergic agonists, such as centrally-acting α2-adrenergic agonists. These compounds act by modulating α2 receptors in certain regions of the brain.

Dexmedetomidine and Other Imidazole Derivatives

In one variation, a composition may include an imidazole such as dexmedetomidine (dextrorotary isomer) or medetomidine (racemic mixture of dextrorotary and levorotary isomers). Dexmedetomidine and medetomidine are 4-[1-(2,3-dimethylphenyl)ethyl]-3H-imidazole, having the formula C13H16N2, as described, for example, in U.S. Pat. No. 4,910,214.

As described in U.S. Pat. No. 6,716,867, the chemical form for dexmedetomidine can be the free base or an acid addition salt. An acid addition salt of dexmedetomidine may be formed, for example, using an inorganic acid (e.g., hydrochloric acid, hydrobromic acid, sulfuric acid, nitric acid, phosphoric acid, etc.), or using an organic acid (e.g., acetic acid, propionic acid, glycolic acid, maltonic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, etc.) or using a fatty acid (e.g., stearic acid, palmitic acid, oleic acid).

Other imidazole derivatives may also be used. As used herein, the term “imidazole derivatives” shall be understood to include, but not be limited to, the imidazole derivatives described in U.S. Pat. No. 4,544,664. Examples of imidazole derivatives described in U.S. Pat. No. 4,544,664 include:

4-[α,α-bis(2-methylphenyl)hydroxymethyl]-5-methylimidazole,

4-[α,α-bis(2-methylphenyl)hydroxymethyl] imidazole, and

4-(α,α-diphenyl)hydroxymethyl-5-methylimidazole.

Imidazole derivatives shall be understood to further include medetomidine (4-[1-(2,3-dimethylphenyl)ethyl]-3H-imidazole), detomidine (4-(2,3-dimethylbenzyl)-imidazole) and 4-[(a-methyl)-2,3-dimethylbenzyl)]imidazole, at least some of which are described, for example, in U.S. Pat. No. 4,670,455.

Clonidine and Clonidine Derivatives

In another variation, a composition may include clonidine or a clonidine derivative. Clonidine, or N-(2,6-dichlorophenyl)-4,5-dihydro-1H-imidazol-2-amine, has the formula C9H9C12N3. Clonidine is described, for example, in U.S. Pat. No. 5,484,607.

Guanfacine and Guanfacine Derivatives

In another variation, a composition may include guanfacine or a guanfacine derivative. Guanfacine, or N-(diaminomethylidene)-2-(2,6-dichlorophenyl)acetamide, has the formula C9H9C12N3O. Examples of guanfacine derivatives which may be used are described, for example, in U.S. Pat. No. 3,632,645.

Additional α2-Adrenergic Agonists

Other examples of α2-adrenergic agonists which may be used include, but are not limited to guanabenz, guanoxabenz, guanethidine, xylazine, tizanidine, and romifidine.

Combination Therapy

In some variations, a combination of compounds may be used, either in the same composition, or in different composition. The different compounds may be administered at the same time or at different times. As an example, a person may take a long-lasting sleep aid prior to going to sleep, but then may take dexmedetomidine after waking up with MOTN insomnia. Alternatively, dexmedetomidine could be coadministered with eszopiclone, with a significant reduction (20 to 50% less) of the amount of eszopiclone used (in comparison to administration of eszopiclone alone). Taken upon retiring, the combination may induce fast sleep onset as result of the dexmedetomidine, as well as provide prolonged sleep duration as a result of the eszopiclone. The combination may also result in minimized eszopiclone side effects because of the reduced dose of eszopiclone.

In certain variations, a combination composition may comprise one or more α2-adrenergic agonists in combination with one or more sedatives or sedative-hypnotics. For example, a combination composition may comprise one or more benzodiazepines, such as alprazolam, diazepam, temazepam (e.g., Restoril®), flunitrazepam (e.g., Rohypnol®), triazolam (e.g., Halcion®), flurazepam (e.g., Dalmane®), nitrazepam (e.g., Mogadon®), and/or midazolam (e.g., Versed®). In some variations, a combination composition may comprise one or more α2-adrenergic agonists in combination with one or more non-benzodiazepines, such as zolpidem (e.g., Ambien®), zaleplon (e.g., Sonata®), zopiclone, eszopiclone (e.g., Lunesta®), ramelteon (e.g., Rozerem®), melatonin, and/or almorexant (e.g., Actelion®), and/or eplivanserin, loxapine, doxepin, 5HT-2a antagonists, or other agents that modify or restore the normal sleep process.

In some variations, a combination composition may comprise one or more α2-adrenergic agonists in combination with one or more anxiolytics, such as buspirone, propranolol, alprazolam (e.g., Xanax®), or clonazepam (e.g., Klonopin®).

In certain variations, a combination composition may comprise one or more α2-adrenergic agonists in combination with one or more secondary therapeutic agents for treating ADHD, such as methylphenidate (e.g., Ritalin®), dextroamphetamine/amphetamine (e.g., Adderall®), dextroamphetamine (e.g., DextroStat®), or atomoxetine (e.g., Strattera®).

### III. PHARMACEUTICALS COMPOSITIONS AND DOSAGE FORMS

The compositions may include one or more α2-adrenergic agonists in any appropriate amount. For example, a composition may comprise an α2-adrenergic agonist in an amount of from about 1% to about 99% by weight of the composition. In one variation, the α2-adrenergic agonist may be included in an amount of from about 0.05% to about 8% by weight of the composition. It is understood that the above dosages are exemplary, and that there may be instances in which higher or lower dosages may be merited.

In some variations, the amount of the α2-adrenergic agonist may be selected to achieve a certain plasma concentration by, for example, aerosol administration (e.g., using the Tempo® inhaler). As an example, the dose range for the α2-adrenergic agonist may be from about 0.01 μg/kg to about 500 μg/kg (e.g., from about 0.2 μg/kg to about 5 μg/kg, or about 2 μg/kg), to be administered in about 1 minute or slower. The corresponding desired plasma concentration may range between about 0.0015 ng/ml to about 600 ng/ml, depending on the general condition of the subject.

In addition to comprising the α2-adrenergic agonist, the compositions may further comprise additional ingredients, such as preservatives, buffers, antioxidants and stabilizers, nonionic wetting or clarifying agents, viscosity-increasing agents, absorption-enhancing agents, pH modifying agents and co-solvents and the like.

For example, inhalation aerosols from dry powder inhalers, nebulizers, vaporizers and pressurized metered dose inhalers typically include excipients or solvents to increase stability or deliverability of these drugs in an aerosol form. As an example, nebulizers generate an aerosol from a liquid, some by breakup of a liquid jet and some by ultrasonic vibration of the liquid with or without a nozzle. Liquid compositions are prepared and stored under aseptic or sterile conditions since they can harbor microorganisms, and thus the use of preservatives is contemplated. Additionally solvents, detergents and other agents may be used to stabilize the drug composition. As another example, for pMDI's, the compositions may be formulated in a canister under pressure with a solvent and propellant mixture, historically chlorofluorocarbons (CFCs), or the replacement hydrofluoroalkanes (HFAs). Upon being dispensed, a jet of the mixture is ejected through a valve and nozzle and the propellant “flashes off”, leaving an aerosol of the compound.

Examples of absorption-enhancing agents include N-acetylcysteine, polyethylene glycols, caffeine, cyclodextrin, glycerol, alkyl saccharides, lipids, lecithin, dimethylsulfoxide, and the like.

Examples of preservatives for use in a solution include polyquaternium-1, benzalkonium chloride, thimerosal, chlorobutanol, methyl paraben, propyl paraben, phenylethyl alcohol, disodium edetate, sorbic acid, benzethonium chloride, and the like. Typically (but not necessarily) such preservatives may be employed at a level from about 0.001% to about 1.0% by weight.

Examples of buffers include boric acid, sodium and potassium bicarbonates, sodium and potassium borates, sodium and potassium carbonates, sodium acetate, sodium biphosphate and the like. In some variations, the buffers may be included in amounts sufficient to maintain the pH of the composition at between about pH 3 and about pH 9 (e.g., between about pH 4 and about pH 7.5).

Suitable antioxidants and stabilizers include ascorbic acid, sodium bisulfite, sodium metabisulfite, sodium thiosulfite, thiourea, caffeine, chromoglycate salts, cyclodextrins and the like. Suitable wetting and clarifying agents include polysorbate 80, polysorbate 20, oleic acid, lecithin and other phospholipids, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The particle size of the drug aerosols may be controlled to provide desired characteristics. For example, when using DPIs, the drug particles may be generated from the bulk drug by attrition processes (e.g., grinding, micronizing, milling, etc.), or by multiphase precipitation processes (e.g., spray drying, solution precipitation, supercritical extraction/precipitation, lyophilization, etc.) to yield powders that may be dispersed in a propellant to obtain an acceptable particle size for delivery to the lungs. As dry powder compositions are prone to aggregation and low flowability, which may result in diminished efficiency, scrupulous attention may be required during milling, blending, powder flow, filling and even administration to ensure that the dry powder aerosols are reliably delivered and have the proper particle size distribution for efficacious delivery to the lungs. In some variations, the powder particles may have a range of diameters from about 0.1 micron to about 10 microns.

### IV. METHODS OF ADMINISTRATION

Administration

The compositions described here may be administered by inhalation using devices such as pressurized metered dose inhalers, breath-actuated inhalers, and dry powder inhalers. In general, a user will first completely exhale. Then the user inhales through the mouthpiece, establishing air flow through the device. For manually actuated devices, the user must actuate the discharge of drug aerosol as they begin to inhale. For breath-actuated devices the device automatically discharges the drug aerosol when the user begins to inhale. The user continues to inhale to fill the lungs to their capacity, and then may hold his or her breath for a period of time to allow the aerosolized drug to settle within the airways deep in the lungs.

The compositions may be administered at a frequency selected to treat the particular disorder. For example, the compositions may be administered once a day, several times a day, weekly, or monthly. The compositions may be administered via a single inhalation or via multiple inhalations. As an example, a person suffering from MOTN insomnia may administer the composition by a single inhalation or a couple of inhalations, and then may not administer the composition again, until the person suffers from another episode of MOTN insomnia.

Dosing Regimens

The dosing regimen employed may depend on a number of factors, such as the type of insomnia or other sleep, anxiety or developmental disorder being treated, the severity of the symptoms, and whether the sleep, anxiety, or developmental disorder is due to an underlying medical condition, and the type of perioperative or non-surgical procedure being performed, if applicable.

The compound may be administered in any appropriate dose. In some variations, the dose may be from about 0.05 μg/kg to about 100 μg/kg (e.g., from about 0.05 μg/kg to about 10 μg/kg, from about 0.1 μg/kg to about 100 μg/kg, from about 10 μg/kg to about 100 μg/kg, from about 0.1 μg/kg to about 10 μg/kg, from about 0.1 μg/kg to about 5 μg/kg, from about 0.2 μg/kg to about 5 μg/kg, from about 0.2 μg/kg to about 4 μg/kg, from about 0.25 μg/kg to about 4 μg/kg, about 2 μg/kg). The dose may be administered from about 1 μg to about 200 μg, and multiple doses may be administered. These examples are suitable for dexmedetomidine. However, other α2-adrenergic agonists may have a higher or a lower potency and therefore require lower or higher dosing regimens respectively.

In some variations, the desired effect may be achieved in about 30 minutes or less (e.g., about 15 minutes or less, about 10 minutes or less, about 5 minutes or less). In certain variations, the desired effect may be achieved within a range from about 0.5 minute to about 15 minutes, such as from about 1 minute to about 5 minutes.

In some variations, the time following administration of the composition at which the peak plasma concentration is attained (Tₘₐₓ) may be about 30 minutes or less (e.g., about 15 minutes or less, about 10 minutes or less, about 5 minutes or less). In certain variations, the plasma concentration of the composition in the subject about 15 minutes or less after administration may be from about 0.0015 ng/mL to about 600 ng/mL.

Sleep Disorders Treated

Methods and compositions described herein may be used to treat one or more sleep disorders including, for example, insomnia, such as transient insomnia, acute insomnia, or chronic insomnia. Middle-of-the-night insomnia may also be treated using the methods and compositions described here.

Anxiety Disorders Treated

The compositions may be used, for example, to treat any of a number of different types of anxiety disorders, either alone or in combination. Examples of anxiety disorders which may be treated using the methods and compositions described here include generalized anxiety disorder, panic disorder, phobias such as agoraphobia, social anxiety disorder, obsessive-compulsive disorder, post-traumatic stress disorder, and separation anxiety disorder. One important form of anxiety is hospital pre-procedural anxiety, such as that experienced by a patient prior a procedure (e.g., injections, blood drawing, insertion of IV cannulas, and MRI scanning). Other anxiety disorders may also be treated.

Developmental Disorders Treated

Methods and compositions described herein may be used to treat one or more developmental disorders including, for example, attention deficit disorders such as attention deficit hyperactivity disorder (ADHD). Pediatric attention deficit disorders may also be treated using the methods and compositions described here.

### V. KITS

The devices and/or compositions described here may be packaged and/or distributed (e.g., to hospitals, clinics, physicians, and/or patients) in an administration kit. Such kits may comprise one or more inhalation devices (e.g., the Tempo® inhaler), and one or more containers (e.g., unit doses or multi-dose containers) of the composition. In some variations, the kit may include one or more devices that are already loaded with the composition. For example, a device may comprise a reservoir that is pre-filled with the composition. Certain variations of kits may include multiple different compositions, and/or multiple different dosages of the same composition. The kit may additionally comprise a carrier or diluent, a case, and/or instructions for operating the appropriate device.

### VI. EXAMPLES

The following examples are intended to be illustrative and not to be limiting.

### Example 1

For pressurized metered dose inhalation, standard 10 ml MDI cans (Presspart Ltd, UK) are crimped with BK357 50 mcl valves (Bespak Ltd, UK), and a suspension (about 100 mg dexmedetomidine citrate in about 7.5 mL HFA 134a) is filled through the valve.

### Example 2

For pressurized metered dose inhalation, standard 10 ml MDI cans (Presspart Ltd, UK) are crimped with BK357 50 mcl valves (Bespak Ltd, UK), and a suspension (about 10 mg dexmedetomidine citrate in about 7.5 mL HFA 134a:227 at a ratio of about 50:50 v/v) is filled through the valve. If tested at 28.3 LPM through an Andersen Cascade Impactor in a BK636 actuator (Bespak Ltd, UK), the fine particle fraction of dexmedetomidine <4.7 microns is anticipated to be >25%.

### Example 3

For pressurized metered dose inhalation, standard 10 ml MDI cans (Presspart Ltd, UK) are crimped with BK357 50 mcl valves (Bespak Ltd, UK), and a suspension (about 45 mg dexmedetomidine citrate in about 7.5 mL HFA 134a:227 at a ratio of 30:70 v/v) is filled through the valve containing 0.1% inhalation grade oleic acid (Super Refined Grade, Croda, UK). If tested at 28.3 LPM through an Andersen Cascade Impactor in a BK636 actuator (Bespak Ltd, UK), the fine particle fraction of dexmedetomidine <4.7 microns is anticipated to be >25%.

### Example 4

Non-Standard 5.9 ml MDI cans (Presspart Ltd, UK) are crimped with BK357 50 mcl valves (Bespak Ltd, UK), and a suspension (about 18 mg dexmedetomidine citrate in about 3 mL HFA 134a:227 at a ratio of about 30:70 v/v) is filled through the valve. If tested at 28.3 LPM through an Andersen Cascade Impactor in a BK636 actuator (Bespak Ltd, UK), the fine particle fraction of dexmedetomidine <4.7 microns is anticipated to be >25%.

### Example 5

For dry powder inhalation, 15 mg portions of a 1% blended composition of dexmedetomidine chloride in lactose (Pharmatose® 200M, DMV, Netherlands) were filled into gelatin capsules and placed into an Aeroliser® DPI. If tested at 60 LPM through an Andersen Cascade Impactor, the fine particle fraction of dexmedetomidine <4.7 microns is anticipated to be >20%.

### Example 6

For a nebulizer solution for inhalation, a 0.15% w/w solution of dexmedetomidine chloride was formulated in a pharmaceutically acceptable buffer for inhalation (0.85% sodium chloride, 0.062% sodium citrate, 0.019% citric acid in water). If tested through a Next Generation Impactor at 15 LPM, the fine particle fraction of dexmedetomidine <4.7 microns is anticipated to be >20%.

While methods, devices, and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

1. A method of inducing arousable sedation in a subject, the method comprising:
administering a therapeutically effective amount of an α₂-adrenergic agonist composition to the subject using a non-injectable route of administration, to initiate an arousable state of sedation within the subject in about 30 minutes or less,
wherein the α₂-adrenergic agonist composition comprises an α₂-adrenergic agonist or a pharmaceutically acceptable salt, hydrate, polymorph, prodrug, ion pair, or metabolite thereof.

2. The method of claim 1, wherein the subject has insomnia, and the arousable state of sedation is induced to treat the insomnia.

3. The method of claim 2, wherein the subject has sleep onset insomnia, and the arousable state of sedation is induced to treat the sleep onset insomnia.

4. The method of claim 2, wherein the subject has middle-of-the-night insomnia, and the arousable state of sedation is induced to treat the middle-of-the-night insomnia.

5. The method of claim 1 wherein said arousable sedation is induced as a perioperative or prediagnostic or a nonsurgical procedure in a clinical setting.

6. The method of claim 1, wherein the α₂-adrenergic agonist composition comprises an α₂-adrenergic agonist selected from the group consisting of dexmedetomidine, clonidine, romifidine, guanfacine, guanabenz, guanoxabenz, guanethidine, xylazine, detomidine, medetomidine, tizanidine, other imidazole derivatives and pharmaceutically acceptable salts, hydrates, polymorphs, prodrugs, ion pairs, and metabolites thereof.

7. The method of claim 6, wherein the α₂-adrenergic agonist composition comprises dexmedetomidine or a derivative of dexmedetomidine or a pharmaceutically acceptable salt, hydrate, polymorph, prodrug, ion pair, or metabolite thereof.

8. The method of claim 6, wherein the α₂-adrenergic agonist composition comprises clonidine or guanfacine or a pharmaceutically acceptable salt, hydrate, polymorph, prodrug, ion pair, or metabolite thereof.

9. The method of claim 1, wherein the arousable state of sedation is initiated within the subject in about 20 minutes or less.

10. The method of claim 1, wherein the α₂-adrenergic agonist composition is administered to the subject via inhalation.

11. The method of claim 10, wherein the α₂-adrenergic agonist composition is administered to the subject via a route of administration selected from the group consisting of oral inhalation and nasal inhalation.

12. The method of claim 11, wherein the α₂-adrenergic agonist composition is administered to the subject using a device selected from the group consisting of a pressurized metered dose inhaler, a breath-actuated metered dose inhaler, a dry power inhaler, and a nebulizer.

13. The method of claim 1, wherein the subject self-administers the α₂-adrenergic agonist composition.

14. The method of claim 1, wherein the Tₘₐₓ for the administration of the α₂-adrenergic agonist is less than about 20 minutes.

15. The method of claim 1, wherein the plasma concentration of the α₂-adrenergic agonist in the subject at 15 minutes or less after administration is in the range of about 0.0015 ng/mL to about 600 ng/mL.

16. The method of claim 1, wherein the α₂-adrenergic agonist composition is administered to the subject at a dosage in the range of about 0.01 μg/kg to about 300 μg/kg.

17. The method of claim 1, wherein the α₂-adrenergic agonist composition is administered to the subject as two separate doses.

18. The method of claim 1, further comprising administering a second therapeutic agent to the subject.

19. The method of claim 18, wherein the second therapeutic agent comprises a sedative or a sedative-hypnotic.

20. The method of claim 18, wherein the second therapeutic agent comprises a benzodiazepine selected from the group consisting of alprazolam, diazepam, temazepam, flunitrazepam, triazolam, flurazepam, nitrazepam and midazolam.

21. The method of claim 18, wherein the second therapeutic agent comprises a non-benzodiazepine selected from the group consisting of zolpidem, zaleplon, zopiclone, eszopiclone, ramelteon, melatonin, a 5-HT₂ₐ antagonist, almorexant, eplivanserin, doxapine, and loxapine.

22. The method of claim 18, wherein the second therapeutic agent is administered to the subject prior to administration of the α₂-adrenergic agonist composition.

23. The method of claim 1 wherein said arousable sedation is induced as a perioperative or prediagnostic or a nonsurgical procedure in a clinical setting.

24. The method of claim 5 wherein the arousable state of sedation is inducted as part of medical procedures selected from the group consisting of MRI, CT scans, wound debridement, abscess drainage, minor skin procedures, difficult vascular access or blood draws, laceration repairs, foreign body removal, endoscopy, colonoscopy, audiology ABR/BAER testing, intra ocular pressure testing, injections of the muscles, bursa, tendons or soft tissue, appliance removal, fracture reduction, ECHO testing, lumbar punctures and bone marrow aspiration procedures, radiology procedures such as nuclear medicine, fluoroscopy, interventional procedures, difficult vascular access, EEG/EMG.SSEP, and dental surgery for children.

25. The method of claim 5, wherein the plasma concentration of the α₂-adrenergic agonist in the subject at 15 minutes or less after administration is in the range of about 0.0015 ng/mL to about 600 ng/mL.

26. The method of claim 5, wherein the α₂-adrenergic agonist composition is administered to the subject at a dosage in the range of about 0.02 μg/kg to about 500 μg/kg.

27. A method of treating an anxiety disorder in a subject, the method comprising:
administering a therapeutically effective amount of an α₂-adrenergic agonist composition to the subject using a non-injectable route of administration, to achieve therapeutic plasma levels in 30 minutes or less, as indicated by a reduction in anxiety symptoms, wherein the α₂-adrenergic agonist composition comprises an α₂-adrenergic agonist or a pharmaceutically acceptable salt, hydrate, polymorph, prodrug, ion pair, or metabolite thereof.

28. The method of claim 27, wherein the anxiety disorder comprises panic disorder.

29. The method of claim 27, wherein the anxiety disorder comprises agoraphobia, social anxiety disorder, obsessive-compulsive disorder, post-traumatic stress disorder, pre-procedural anxiety and/or separation anxiety disorder.

30. The method of claim 27, wherein the α₂-adrenergic agonist composition comprises an α₂-adrenergic agonist selected from the group consisting of dexmedetomidine, clonidine, romifidine, guanfacine, guanabenz, guanoxabenz, guanethidine, xylazine, detomidine, medetomidine, tizanidine, other imidazole derivatives and pharmaceutically acceptable salts, hydrates, polymorphs, prodrugs, ion pairs, and metabolites thereof.

31. The method of claim 30, wherein the α₂-adrenergic agonist composition comprises dexmedetomidine or a derivative of dexmedetomidine or a pharmaceutically acceptable salt, hydrate, polymorph, prodrug, ion pair, or metabolite thereof.

32. The method of claim 30, wherein the α₂-adrenergic agonist composition comprises clonidine or a pharmaceutically acceptable salt, hydrate, polymorph, prodrug, ion pair, or metabolite thereof.

33. The method of claim 27, wherein reduction in anxiety levels is achieved within the subject in about 20 minutes or less.

34. The method of claim 27, wherein the α₂-adrenergic agonist composition is administered to the subject via inhalation.

35. The method of claim 34, wherein the α₂-adrenergic agonist composition is administered to the subject via a route of administration selected from the group consisting of oral inhalation and nasal inhalation.

36. The method of claim 35, wherein the α₂-adrenergic agonist composition is administered to the subject using a device selected from the group consisting of a pressurized metered dose inhaler, a breath-actuated metered dose inhaler, a dry power inhaler, and a nebulizer.

37. The method of claim 27, wherein the subject self-administers the α₂-adrenergic agonist composition.

38. The method of claim 27, wherein the Tₘₐₓ for the administration of the α₂-adrenergic agonist is less than about 20 minutes.

39. The method of claim 27, wherein the plasma concentration of the α₂-adrenergic agonist in the subject at 15 minutes or less after administration is from about 0.0015 ng/mL to about 600 ng/mL.

40. The method of claim 27, wherein the α₂-adrenergic agonist composition is administered to the subject at a dosage of from about 0.01 μg/kg to about 500 μg/kg.

41. The method of claim 27, wherein the α₂-adrenergic agonist composition is administered to the subject as two separate doses.

42. The method of claim 27, further comprising administering a second therapeutic agent to the subject.

43. The method of claim 42, wherein the second therapeutic agent comprises an anxiolytic agent selected from the group consisting of buspirone, propranolol, alprazolam, clonazepam, doxepine, loxapine, and a 5HT-2a antagonist.

44. A method of treating ADHD in a subject, the method comprising:
administering a therapeutically effective amount of an α₂-adrenergic agonist composition to the subject using a non-injectable route of administration, to achieve a reduction in hyperactivity or associated symptoms thereof in 30 minutes or less,
wherein the α₂-adrenergic agonist composition comprises an α₂-adrenergic agonist or a pharmaceutically acceptable salt, hydrate, polymorph, prodrug, ion pair, or metabolite thereof.

45. The method of claim 44, wherein the α₂-adrenergic agonist composition comprises an α₂-adrenergic agonist selected from the group consisting of dexmedetomidine, clonidine, romifidine, guanfacine, guanabenz, guanoxabenz, guanethidine, xylazine, detomidine, medetomidine, tizanidine, other imidazole derivatives and pharmaceutically acceptable salts, hydrates, polymorphs, prodrugs, ion pairs, and metabolites thereof.

46. The method of claim 45, wherein the α₂-adrenergic agonist composition comprises dexmedetomidine or a derivative of dexmedetomidine or a pharmaceutically acceptable salt, hydrate, polymorph, prodrug, ion pair, or metabolite thereof.

47. The method of claim 45, wherein the α₂-adrenergic agonist composition comprises clonidine or a pharmaceutically acceptable salt, hydrate, polymorph, prodrug, ion pair, or metabolite thereof.

48. The method of claim 45, wherein the α₂-adrenergic agonist composition comprises guanfacine or a pharmaceutically acceptable salt, hydrate, polymorph, prodrug, ion pair, or metabolite thereof.

49. The method of claim 44, wherein a reduction in hyperactivity or associated symptoms thereof is achieved within the subject in about 20 minutes or less.

50. The method of claim 44, wherein the α₂-adrenergic agonist composition is administered to the subject via inhalation.

51. The method of claim 50, wherein the α₂-adrenergic agonist composition is administered to the subject via a route of administration selected from the group consisting of oral inhalation and nasal inhalation.

52. The method of claim 51, wherein the α₂-adrenergic agonist composition is administered to the subject using a device selected from the group consisting of a pressurized metered dose inhaler, a breath-actuated metered dose inhaler, a dry power inhaler, and a nebulizer.

53. The method of claim 44, wherein the subject self-administers the α₂-adrenergic agonist composition.

54. The method of claim 44, wherein the Tₘₐₓ for the administration of the α₂-adrenergic agonist is less than about 20 minutes.

55. The method of claim 44, wherein the plasma concentration of the α₂-adrenergic agonist in the subject at 15 minutes or less after administration is in the range of about 0.0015 ng/mL to about 600 ng/mL.

56. The method of claim 44, wherein the α₂-adrenergic agonist composition is administered to the subject at a dosage in the range of about 0.01 μg/kg to about 500 μg/kg.

57. The method of claim 44, wherein the α₂-adrenergic agonist composition is administered to the subject as two separate doses.

58. The method of claim 44, further comprising administering a second therapeutic agent to the subject.
